(19) United States
(12) Patent Application Publication
(10) Pub. No.: US 2002/0062491 A1
(43) Pub. Date: May 23, 2002

(54) Method of producing primary hepatocellular carcinoma-bearing model animals

(75) Inventor: Shun-Ichiro Ishii, Hyogo (JP)

Correspondence Address: SUGHRUE, MION, ZINN, MACPEAK & SEAS, PLLC, Suite 800, 2100 Pennsylvania Avenue, N.W., Washington, DC, 20037-3213, US

(73) Assignee: WELFIDE CORPORATION

(21) Appl. No.: 09/955,092

(22) Filed: Sep. 19, 2001

(30) Foreign Application Priority Data
Sep. 20, 2000 (JP) HEI2000-284635

(51) Int. Cl. A01K 67/00

(52) U.S. Cl. 800/10

(57) ABSTRACT

A method for producing primary hepatocellular carcinoma-bearing model animals is provided and is made easily and surely in a large scale. The model animals are effectively utilized for a method of diagnosis and therapy of primary hepatocellular carcinoma.

### TECHNICAL FIELD

The present invention relates to a production of primary hepatocellular carcinoma-bearing model animals and to the model animals produced thereby. More particularly, the present invention relates to a production of primary hepatocellular carcinoma-bearing model animals, characterized by being selected and subjected to administrating diethylnitrosoamine, breeding with a choline-deficient amino acid diet, and a normal diet, in a combined manner under a predetermined condition and also relates to the model animals produced as such.

### BACKGROUND OF THE INVENTION

It has been known that, in advance of a occurrence of hepatocellular carcinoma, many progressive steps and processes are existed in carcinoma and occurrence thereof is resulted after a period of time for showing preneoplastic lesions. With regard to a method for producing carcinoma-bearing model animals, it is a conventional method where known carcinogen is mixed with drinking water or with feed, and is administered for a long period of time but said method is hardly adopted as for producing the primary hepatocellular carcinoma-hearing model animals as an object of hepatocellular carcinoma, because of too heavy labor needed, and many risks caused.

When a carcinogen screening system, such as a medium-term carcinogenesis bioassay system (Exp. Toxic Pathol., 48, p. 113-119, 1996) on the basis of a two-step carcinogenesis theory (a theory that carcinogenesis comprises two steps, i.e. initiation and promotion) [Hosp. Pract. (Off Ed.), 18(7), p. 101-113, 1983] is utilized, it is possible to produce preneoplastic lesions within a short period of time (e.g., about eight weeks) by a combination of administration of carcinogen and partial hepatectomy (PH), and soon, although hepatocellular carcinoma is not produced thereby. Therefore, the above method is unable to be adopted as a method for producing a primary hepatocellular carcinoma-bearing model animals.

### SUMMARY OF THE INVENTION

On comparing incidences of hepatocellular carcinoma internationally, in recent years, although those show high level in Asia, including Japan, and Africa, an augmentation is predicted, in future, in Europe and America, as well. The main cause of generation of primary hepatocellular carcinoma is infection of hepatitis virus of type B and type C and in most cases, it has been known, to be accompanied with cirrhosis. Therefore, with regard to the primary hepatocellular carcinoma-bearing model animals, there is a demand that the frequency of incidence of the resulting carcinoma is high and that symptoms including the source of generation of carcinoma (which is to be cirrhosis) which are quite similar to clinical ones, are achieved and, in addition, it is desired that the method for producing the said animals is as simple as possible.

An object of the present invention is to provide a method for producing primary hepatocellular carcinoma-bearing model animals which solve above-mentioned problems.

As the results of studies on a synergism of various treatments which have been adopted in the carcinogen screening system such as a medium-term carcinogenesis bioassay system, etc., the inventor found that the desired object can be efficiently achieved by a combination of administration of diethylnitrosoamine (DEN), breeding with a (CDAA) choline-deficient amino acid diet and breeding with a normal diet, under a predetermined condition and accomplished the present invention. Thus, the present invention comprises the followings:

1. A method for producing primary hepatocellular carcinoma-bearing model animals comprising the following steps;

1) a step for selecting species and strain of model animals in sensitivity on a promotion action for CDAA diet and an sensitivity to the initiation action for DEN, as indexes;

2) a step for administering with DEN under breeding with the CDAA diet to the selected animals and progressing the carcinogenesis and

3) a step for establishing hepatocellular carcinoma-bearing model animals by switching the CDAA diet to a normal diet.

2. The method according to the above 1, wherein a time period for breeding with the CDAA diet, is at least 64 weeks as a whole.

3. The method according to the above 1, wherein a time period for breeding with the normal diet, is at least 8 weeks.

4. The method according to the above 1, wherein the administration of DEN is a one-time administration of carcinogenically effective dose for liver of the selected animals.

5. The method according to the above 4, wherein the administration of DEN is made after a preliminary breeding time period for a breeding with the CDAA diet.

6. The method according to the above 5, wherein the preliminary breeding time period is about one week.

7. The method according to the above 1, wherein the animal species is rat.

8. The method according to the above 7, wherein a strain of the rat is F344 (Fischer).

9. The method according to the above 1, wherein the breeding time period with the CDAA diet is at least 64 weeks, the breeding time period with the normal diet is at least 8 weeks and the administration of DEN is a one-time administration of carcinogenically effective dose for a liver of the selected animals.

10. The method according to the above 9, wherein the selected animals are rats of an F344 (Fischer) strain.

11. The method according to the above 1, the model animals bearing a different degrees of a hepatocellular carcinoma is produced by controlling the time period for bleeding a choline-deficient amino acid diet after administration of diethylnitrosoamine.

12. The method according to the above 11, wherein a time period for bleeding the normal diet is at least 8 weeks.

13. A method for diagnosis/therapy of primary hepatocellular carcinoma using the primary hepatocellular carcinoma-bearing model animals produced by the method of any of proceeding the above 1 to 12.

14. A method of screening diagnostic/preventive/therapeutic agent for primary hepatocellular carcinoma using the primary hepatocellular carcinoma-bearing model animals produced by the method of any of proceeding the above 1 to 12.

15. Primary hepatocellular carcinoma-bearing model animals produced by the method of the above 1.

16. The model animals according to the above 15, wherein said animals bear a different degree of a hepatocellular carcinoma.

17. A novel agent for diagnosis/therapy/prevention for primary hepatocellular carcinoma prepared by the method of the above 14.

### BRIEF DESCRIPTION OF THE DRAWINGS

FIG. 1 is a drawing which shows a dieting process to the test animals in Experimental Example 1 and

FIG. 2 shows foci images of livers of six samples of groups 2-9 in Experimental Example 1.

### DESCRIPTION OF THE PREFERRED EMBODIMENTS

Species and strains of the model animals are selected in sensitivity to the promotion action for a CDAA diet and initiation action for DEN, as indexes. Preferred model animals are rats of an F344 (Fischer) strain, which are available from Charles River Japan Inc., etc.

Diethylnitrosoamine (DEN) has been known to have carcinogenicity to liver of rats, and, in the present invention, DEN is used as an initiator. DEN is available as a commercial product (manufactured by Sigma Chemical Co.), which dose is made not less than about 20 mg/kg body weight. If the dose is less than that, probably a carcinogenic effect may not be fully achieved. A stage for administrating is preferably about one week after the start of breeding with the CDAA diet. In the administration, a pure DEN solution is diluted with a physiological saline to an appropriate concentration and is administered preferably intraperitoneally for one to plural (2 to 4) times.

A CDAA diet is a synthetic diet mainly composed/constituted with various amino acids except choline. This diet was invented by Nara Medical University (Cancer Research, 15, 52 (18), p. 5042-5045, 1992) and is available from Dyes Inc., U. S. A. (#518753). Unlike the common commercially available semi-purified choline-deficient diets, said diet is a characterised by not containing choline at all.

Composition of a CDAA diet (grams in each ingredient per kg of the CDAA diet) is as shown in Table 1.

Since a CDAA diet does not contain choline at all, a promotion action, due to a deficiency of choline can be strongly achieved. Further, as a result of breeding with this diet, a liver of the model animals gradually becomes cirrhosis where triglycerides are potently accumulated in hepatocytes. Since the diet is just freely taken by the animals during the breeding period, there is no need of labor such as repeated administration of carcinogen at all. During the breeding period with the CDAA diet, hepatocytes progress to canceration due to the initiation and promotion effects of DEN and the CDAA diet.

It has been confirmed by confirmation of canceration of cells that, when male rats of F344 strain are used and the breeding period with a normal diet is made constant (about 8 weeks), the canceration degree of the model animals produced by the present invention is mostly decided by the breeding period with the CDAA diet before the breeding with the normal diet. When the breeding period with the CDAA diet is about 24 weeks, the resulting tumor is to an extent of hyperplastic nodule (altered hepatocellular foci) which is a kind of preneoplastic lesions and hardly proceeds to canceration. When the breeding period with the CDAA diet is about 32 weeks, the outcome is that, in some animals, canceration is observed. In order to achieve hepatocellular carcinoma in a high frequency when rats of F344 strain are used as a carcinoma-bearing model animals, it is necessary that the breeding period with a CDAA diet is required for about 64 weeks or longer. During the breeding period, the CDAA diet is made freely taken by the object model animals.

There is no particular limitation for the normal diet, and a common breeding diets commercially available for rats, such as CRF-1 (Oriental Yeast Co., Ltd.) etc., may be used. After breeding with the CDAA diet for a predetermined period, the diet is entirely switched to the normal diet and the breeding is carried out for about 8 weeks or longer. During the breeding period, the normal diet is freely taken by the object model animals.

To be specific, a composition of CRF-1 (in gram numbers of each ingredient per kg of CRF-1) is as follows.

Water 8.1, crude protein 22.6, crude lipid 5.6, crude ash 6.6, crude fiber 3.3, soluble matter containing no nitrogen 53.8; calorie: 356 kcal.

With regard to vitamins: A 3783 IU, D₃ 503 IU, E 21.2 mg, B₁ 4.44 mg, B₂ 3.06 mg, C 14 mg, B₆ 1.26 mg, B₁₂ 12.2 μg, inositol 431 mg, biotin 27.8 μg, pantothenic acid 7.07 mg, niacin 14.6 mg, choline 0.31 and folic acid 0.25 mg.

With regard to minerals, calcium 1.27, phosphorus 0.84, magnesium 0.25, sodium 0.32, potassium 0.85, iron 16.6 mg, aluminum 4.2 mg, copper 0.92 mg, zinc 6.94 mg, cobalt 0.28 mg, manganese 7.78 mg, chlorine 0.58 and iodine 0.93 mg.

With regard to amino acids, isoleucine 0.86, leucine 1.7, lysinel 1.28, methionine 0.48, cystine 0.35, phenylalanine 0.97, tyrosine 0.66, threonine 0.88, tryptophan 0.28, valine 1.08, argininel 1.36, histidine 0.57, alaninel 1.20, asparticacidl 1.97, glutamic acid 3.67, glycine 1.12, proline 1.26 and serine 1.06 (all of amino acids are of L-type).

Others, phospholipids 906 mg, cholesterol 66 mg and triglycerides 4.2 mg.

As the breeding with the CDAA diet proceeds, hepatocytes of the model animals progress to canceration. At the same time, triglycerides are potently accumulated in the hepatocytes and, therefore, this stage is believed to be a special diseased state in a clinical viewpoint. Therefore, the CDAA diet was switched to the normal diet and breeding is carried out for more 8 weeks or longer, whereupon most of the accumulated triglycerides are naturally removed from hepatocytes. At the same time, the cells cancerated during the breeding period with the CDAA diet are not retrogressed even during the breeding period with the normal diet, but still grow. Accordingly, by way of a breeding period with the normal diet, there is obtained hepatocellular carcinoma with various degrees of differentiation where cirrhosis with less accumulation of triglycerides is a generation source.

As such, primary hepatocellular carcinoma-bearing model animals produced according to the method of the present invention express the hepatocellular carcinoma where cirrhosis is a generating source in a high frequency.

As mentioned above, primary hepatocellular carcinoma-bearing model animals can be easily and surely produced in a large scale by the producing method of the present invention and it is also possible to commercially provide the said model animals. The primary hepatocellular carcinoma-bearing model animals produced by the present invention can be utilized for a method of diagnosis and therapy of primary hepatocellular carcinoma and for screening diagnostic, preventives and therapeutic agents thereof by common means. The hepatocellular carcinoma-bearing model animals were actually used for evaluation of imaging ability of MRI contrast medium for hepatocellular carcinoma whereupon hepatocelular carcinoma of various degrees of differentiation was observed at the same time and the animals have been confirmed to be quite useful as model animals.

Thus, the primary hepatocellular carcinoma-bearing model animals in accordance with the present invention can be the model animals which are suitable for the study of hepatic image diagnosis such as MRI and CT and also for the research and development of contrast media and anticancer agents. In addition, since a series of changes from preneoplastic lesions to hepatocellular carcinoma in various degrees of differentiation can be obtained, it is expected that useful samples are provided for the studies of histopathology of hepatocellular carcinoma or for the studies of molecular cytology for mechanism of generation and progress of carcinoma.

### EXAMPLES

In order to illustrate the present invention in more detail, there will be provided Experimental Examples and Examples as hereunder although the present invention is not limited by them at all.

### Experimental Example 1

In order to detect suitable system for the production of hepatocellular carcinoma bearing model animal, the male rats (four weeks old) of an F344 strain were classified into various groups where administration of DEN (intraperitoneal), administration of phenobarbital (PB) (intrapertoneal), treatment of partial hepatectomy (PH) and breeding with a CDAA diet were combined and the cancerated effects during 8 weeks were compared. The result was that, with regard to the focal area, the group administered with the CDAA diet and DEN shows a significant increase as compared with the group administered with the CDAA diet without DEN and the group administered with the normal diet and DEN, whereby the CDAA diet achieved a strong promoting action to the initiation by DEN. On the other hand, the effect of further PB administration and PH treatment to the DEN-administered animals to which the CDAA diet was given was not clear. In addition, breeding with the CDAA diet potently accumulated the triglycerides in hepatocytes. From those results, for the original object, there was suggested a possibility that a combination of administration of DEN, breeding with a CDAA diet and a normal diet is useful. Further, in order to check the effect of administration of DEN in the production of the hepatocellular carcinoma-bearing model rats, expression frequency of hepatocellular carcinoma and degree of malignancy of the preneoplastic lesions were compared in the liver between the group which was administered with DEN and the group which was not administered with DEN at the stage of about 54 weeks in the breeding with a CDAA diet and, as a result, apparent increases were noted in the DEN-administered group in both terms of expression frequency and degree of malignancy.

As hereunder, detailed experimental results will be shown.

Materials and Methods

Compounds

Diethylnitrosoamine (DEN, Sigma Chemical Co., USA) and phenobarbital (PB, Wako Pure Chemical Industries Ltd., Osaka) were used as an initiater and promoter, respectively. Saline (Otsuka Pharmaceutical Factory, Inc., Tokushima) was used as vehicle for both compounds.

Animals

The eighty six male 4-week-old Fischer 344 rats used in this study were obtained from Charles River Japan, Inc. (Kanagawa). Grouping of the rats was performed according to the latest body weight. The animals were housed, two/cage in a climate-controlled animal room at temperature of 24±3° C., relative humidity of 55±15%, and ventilation rate of 10-20 times/h with 12-hour illumination, and allowed free access to the prescribed diets in every group and tap water.

Groups and treatment methods

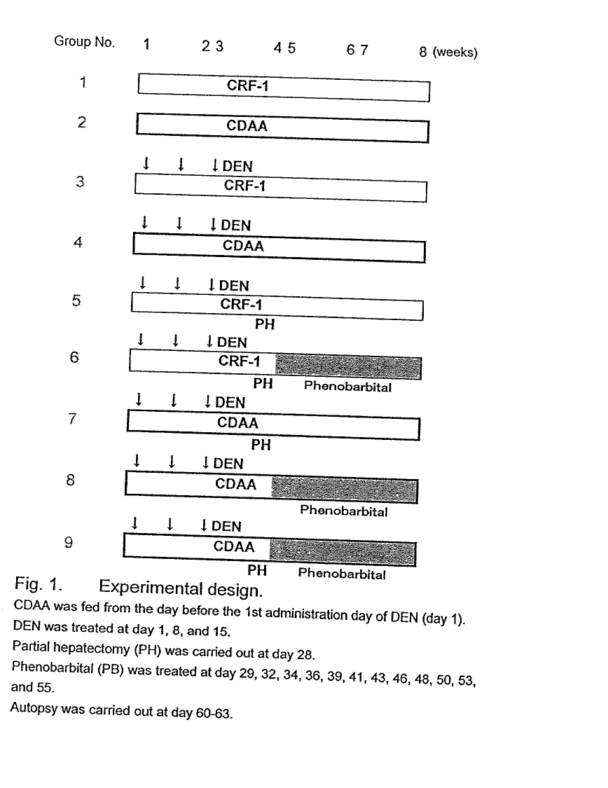

Experimental design is shown in FIG. 1. To the animals in group 1, only a CRF-1 basal diet (Oriental Yest Co., Ltd., Tokyo) was fed. To the ones in group 2, only a CDAA (Dyets Inc., USA) diet was fed. The CDAA diet, whose composition is shown in Table 1, was fed from the day before the 1^{st} administration day of DEN (day 1) and was stored under refrigeration until use. To the ones in group 3, a CRF-1 basal diet was fed and DEN was treated. The dose of DEN was set at 95 mg/kg body weight (2 ml/kg body weight) and rats were intraperitoneally treated once a week for the first three weeks (day 1, 8 and 15). To the ones in group 4, a CDAA diet was fed and DEN was treated. To the ones in group 5, a CRF-1 basal diet was fed and given DEN and PH. In the partial hepatectomy (PH), the left lobe and the right and left sublobes of the median lobe of the liver were excised. PH was carried out on the day before the first administration of PB (day 28). To the ones in group 6, a CRF-1 basal diet was fed and given DEN, PH, and PB. The dose of PB was set at 60 mg/kg body weight (2 ml/kg body weight) and rats were intraperitoneally treated three times a week from week 5 (day 29, 32, 34, 36, 39, 41, 43, 46, 48, 50, 53, and 55). To the ones in group 7, a CDAA diet was fed and given DEN and PH. To the ones in group 8, a CDAA diet was fed and given DEN and PB. To the ones in group 9, a CDAA diet was fed and given DEN, PH, and PB. This experiment was started on Apr. 10, 1997.

Blood chemistry examination

At autopsy (day 60-63), blood was collected from the ventral aorta and alkaline phosphatase (ALP, p-NPP substrate method), glutamic-oxaloacetictransaminase (GOT, Henry altered method), glutamic-pyrvic transaminase (GPT, Henry altered method), γ-glutamyl transpeptidase (γ-GTP, γ-Glu-pNA substrate method), total cholesterol (TCH, CHE-COD-POD method), triglyceride (TG, LPL-GK-GPO-POD method), and free fatty acid (FFA, ACS-MK-PK-LDH method) in serum measured using an autoanalyzer (AU550, Olympus Optical Co., Ltd., Tokyo).

Pathological examination

At eight weeks (day 60-63), autopsy was performed after the animals were sacrificed by exsanguination under ether anesthesia. After the liver had been excised and fixed in 10% neutral buffered formalin fixative, paraffin sections and H.E.-stained specimens were prepared and microscopic examination was carried out.

Immunohistochemical staining using polyclonal antibodies against GST-P was applied to facilitate quantitative evaluation of altered hepatocellular foci. Paraffin sections were stained immunohistochemically using the labeled streptavidin-biotin-peroxidase complex (LSAB) method for binding of rabbit anti-rat GST-Yp polyclonal antibody (1:1000; BIO PREP, IRELAND). The analytical procedure for the GST-P-stained specimens was as follows. Photographs (x10) of GST-P-stained specimens of whole cross-section of liver were taken. Photographs were jointed to construct complete cross-sectional image. GST-P-positive foci (hereinafter foci) were painted out with felt pen. Cross-sectional images were processed by Image Scanner (GT-6500, SEIKO EPSONCO., Ltd., Nagano) (using EpScan Mac). The area of each focus and total cross-sectional area of each image were measured using Adobe Photoshop and NIH image. Number of foci per unit area and focal area per unit area were calculated from data on number of foci, mean focal area, total focal area, and total organ area.

Statistical Evaluation

All examination data were statistically evaluated by F-t test. First, equal dispersion was tested by F test (significance level 5%). Student-t test was carried out where population variance was judged to be equal, and Welch-t test where it was judged different. Bilateral significance level was set at 5% and 1%.

Results

Survival, final body weight, and liver weight

Survival, final body weight, and liver weight are shown in Table 2. In the CDAA+DEN-treated groups (7 and 9), four animals died after PH. Final body weight was reduced in the CDAA-only group (2) in comparison with the CRF-1-only group (1), and was further reduced in the CDAA+DEN-treated groups (4, 7, 8, and 9) in comparison with the CDAA-only group (2). Liver weight was markedly elevated in the CDAA-only group (2) in comparison with the CRF-1-only group (1), and was further elevated in the CDAA+DEN-treated, but not PH-treated groups (4 and 8) in comparison with the CDAA-only group (2).

Autopsy and histopathological findings in liver, and blood chemistry examination

In the CDAA-treated groups (2, 4, 7, 8 and 9), a pale enlarged liver with a rough surface was observed in almost all animals. Histopathological findings of H.E.-stained specimens are shown in Table 3. In group 1 (CRF-1), no marked change was observed. Findings in group 2 (CDAA) not observed in group 1 (CRF-1) were fibrosis, severe vacuolar degeneration, oval cell formation, and altered hepatocellular foci. Findings in group 3 (CRF-1+DEN) not observed in group 1 (CRF-1) were slight vacuolar degeneration and altered hepatocellular foci. In group 4 (CDAA+DEN), fibrosis was more marked and oval cells more numerous than in group 2 (CDAA). The altered hepatocellular foci appeared pseudocircular separated by fiber. Almost all animals showed such changes to a severe degree. In group 5 (CRF-1+DEN+PH), altered hepatocellular foci were more marked than in group 3 (CRF-1+DEN). Group 6 (CRF-1+DEN+PH+PB) showed almost the same findings as group 3 (CRF-1+DEN). In group 7 (CDAA+DEN+PH), oval cells were slightly more numerous than in group 4 (CDAA+DEN). In groups 8 (CDAA+DEN+PB) and 9 (CDAA+DEN+PH+PB), findings were almost the same as in group 4 (CDAA+DEN).

In blood chemistry examination, in the CDAA-treated groups (2, 4, 7, 8, and 9), GOT, GPT, γ-GTP, and FFA were elevated and TG reduced in comparison with group 1 (CRF-1). In the CDAA+DEN-treated groups (4, 7, 8, and 9), γ-GTP was markedly elevated in comparison with group 2 (CDAA).

Analysis of GST-P-stained specimens

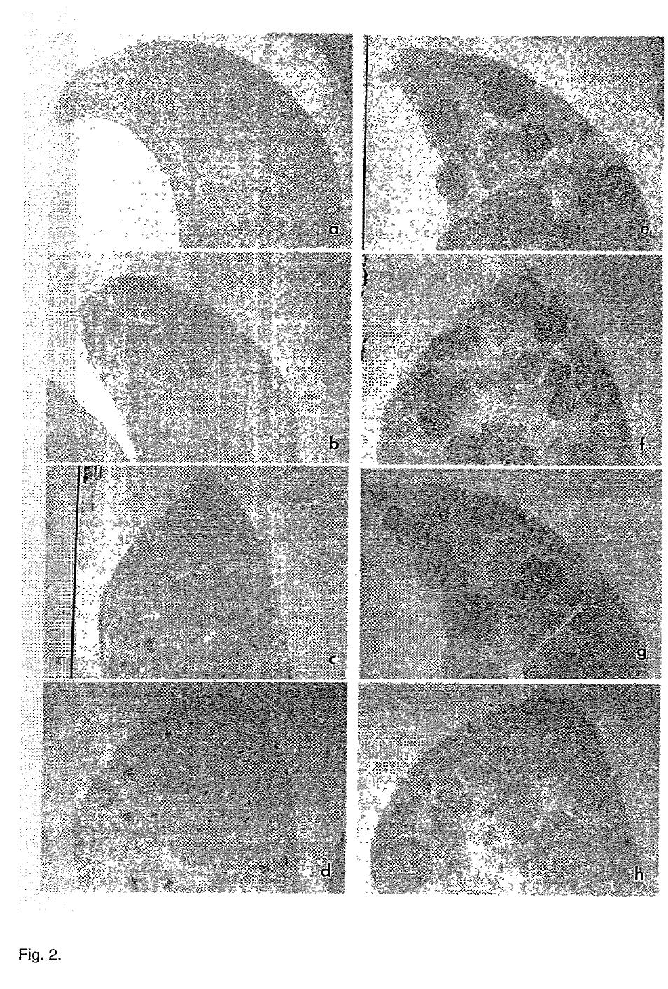

Figures for focal area are shown in Table 4, and for number of foci by size in Table 5. In group 1 (CRF-1), no foci were observed. In groups 2 (CDAA) and 3 (CRF-1+DEN), foci were observed (FIG. 2a,b). In group 4 (CDAA+DEN), in comparison with group 2 (CDAA), number of foci and focal area were elevated, as did number of foci in all size groups (FIG. 2e). In group 5 (CRF-1+DEN+PH), in comparison with group 3 (CRF-1+DEN), focal areas were elevated, as did number of foci of size 0.03-1.0 mm² (FIG. 2c). In group 6 (CRF-1+DEN+PH+PB), in comparison with group 3 (CRF-1+DEN), number of foci and focal area were elevated, did number of foci of size 0-1.0 mm² (FIG. 2d). In group 7 CDAA+DEN+PH), in comparison with group 4 (CDAA+DEN), number foci of size 0-0.03 mm² was reduced (FIG. 2f). In groups 8 CDAA+DEN+PB) and 9 (CDAA+DEN+PH+PB), in comparison with group 4 (CDAA+DEN), no change was observed (FIG. 2g,h).

Summary of the result

Focal area was markedly elevated in the CDAA+DEN-treated group (4) in comparison with the CDAA-only group (2) or the CRF-1+DEN group (3), but was not further elevated in the groups treated with PH, PB or PH+PB in addition to CDAA+DEN (7, 8, and 9). The effect of additional PH and PB treatment was not clear in the CDAA+DEN-treated groups (4, 7, 8, and 9). These findings were taken as showing that CDAA diet had a strong promotion effect in cells initiated by DEN. In the CDAA+DEN-treated groups (4, 7, 8, and 9), histopathological examination showed marked fibrosis, and blood chemistry examination showed marked elevation of γ-GTP in comparison with the CDAA-only group (2). The marked fibrosis was thought to be induced by severe hepatocyte necrosis and an indication of strong cytotoxicity by synergistic effects of DEN and CDAA diet. Since a marked increase of GST-P-positive foci was observed in the CDAA+DEN-treated groups (4, 7, 8, and 9), increase in blood γ-GTP was thought to be a change related to the formation of hepatocellular enzyme-altered foci. Meanwhile, in the CRF-1+DEN-treated animals, the additional PB and PH treatment elevated the number of small foci, but slightly did the focal area. Since focal area in the groups treated with PH or PH+PB in addition to CRF-1+DEN (5, 6) showed much lower values than in the CDAA+DEN group (4), the promotion effect of PH and PB was thought to be much weaker than that of the CDAA diet.

These results showed that CDAA diet was more promotive than the PB and PH treatment in inducing hepatocellular foci formation initiated by DEN in rats, and the additional PB and PH treatment could not be promotive furthermore in the CDAA+DEN-treated animals.

### Example 1

In the production of hepatocellular carcinoma-bearing model rats by administration of DEN and breeding with a CDAA diet, the effect of the breeding with a normal diet after the breeding with the CDAA diet was studied. The F344 male rats (5 weeks old) were bred with the CDAA diet for one week, DEN was intraperitoneally administered one time at the dose of 20 mg/kg body weight, bred with the CDAA diet for 64 weeks as a whole and then bread with the normal diet (CRF-1) for 8 weeks or more to produce the primary hepatocellular carcinoma-bearing model animals of the present invention. As a result, it was confirmed that most of the triglycerides accumulated in the hepatocytes were removed by breeding with the normal diet for 8 weeks or more after breeding with the CDAA diet. Incidentally, accumulation of the triglycerides in hepatocytes as a result of breeding with the CDAA diet is presumed to be the fact that, due to the deficiency of choline, lecithin necessary for transportation of the triglycerides in hepatocytes is not able to be synthesized. Accordingly, it is believed that, when that is switched to breeding with the normal diet whereby choline necessary for the synthesis of lecithin is provided, the transportation ability for the triglycerides is revived and the triglycerides can be quickly removed from the inside of the hepatocytes.

### Example 2

F344 male rats (five weeks old) were bred for one week with a CDAA diet, then DEN was intraperitoneally administered one time at the dose of 20 mg/kg body weight, breeding with the CDAA diet was conducted for 64 weeks as a whole and breeding with a normal diet (CRF-1) for 8 weeks or longer was conducted whereupon the primary hepatocellular carcinoma-bearing model animals of the present invention were produced. The said animals were subjected to the histopathological examination for liver whereupon hepatocellular carcinoma of various degrees of differentiation and preneoplastic lesions were observed in a high frequency.

TABLE 1
Compositions of Choline-deficient L-amino acid-defined (CDAA)
Diet

| Ingredient | (g/kg diet) |
|---|---|
| L-Arginine | 12.7 |
| L-Histidine | 3.4 |
| L-Lysine HCl | 9.1 |
| L-Tyrosine | 5.7 |
| L-Tryptophan | 1.8 |
| L-Phenylalanine | 7.3 |
| L-Methionine | 1.7 |
| L-Cystine | 3.7 |
| L-Threonine | 4.6 |
| L-Leucine | 10.5 |
| L-Isoleucine | 6.1 |
| L-Valine | 6.3 |
| Glycine | 6.2 |
| L-Proline | 7.6 |
| L-Glutamic acid | 28.9 |
| L-Alanine | 5.1 |
| L-Asparatic acid | 15.8 |
| L-Serine | 7.2 |
| Corn strarch | 100 |
| Dextrin | 100 |
| Cellulose | 50 |
| Sucrose | 406.67 |
| Sodium bicarbonate | 4.3 |
| Corn oil | 50 |
| Primex | 100 |
| Modified AIN-76 salt mix | 35 |
| AIN-76A vitamin mix | 10 |
| Ferric citrate | 0.33 |

TABLE 2
Body Weight and Liver Weight

| | | | | | Number of animals | | Body weight (day 57) | Liver weight |
|---|---|---|---|---|---|---|---|---|
| Group | DEN | CDAA | PH | PB | at Start | at Autopsy | (g) | (g) |
| 1 | − | − | − | − | 10 | 10 | 281.97 ± 14.09 CT | 6.856 ± 0440 CT |
| 2 | − | + | − | − | 10 | 10 | 222.86 ± 9.21 ** CT | 11.889 ± 1183 ** CT |
| 3 | + | − | − | − | 10 | 10 | 257.52 ± 5.11 ** CT | 6387 ± 0226 ** CT |
| 4 | + | + | − | − | 10 | 10 | 192.55 ± 7.82 ** ** CT | 13430 ± 1367 ** ** CT |
| 5 | + | − | + | − | 4 | 4 | 263.85 ± 3.96 ** * | 6335 ± 0234 * . |
| 6 | + | − | + | + | 4 | 4 | 247.15 ± 4.10 ** ** | 6225 ± 0153 * . |
| 7 | + | + | + | − | 14 | 10# | 169.28 ± 14.23 ** ** | 9449 ± 2352 * ** |
| 8 | + | + | − | + | 10 | 10 | 190.32 ± 7.21 ** . | 14118 ± 1370 ** . |
| 9 | + | + | + | + | 14 | 10# | 175.32 ± 12.33 ** ** | 10365 ± 1011 * ** |

DEN diethylnitrosamine, CDAA: choline deficient L-amino acid defined diet. PH, partial hepatectomy, PB: phenobarbital, CDAA(−). CRF-1 was fed to animals.
#Differences from the start means number of the dead after PH.
**,*Significantly different from control (CT) (**:p < 0.01, *:p < 0.05). -: Not signficantly different

TABLE 3
Histopathological Findings of Liver

| | Group | 1 | 2 | 3 | 4 | 5 | 6 | 7 | 8 | 9 |
|---|---|---|---|---|---|---|---|---|---|---|
| | DEN | − | − | + | + | + | + | + | + | + |
| | CDAA | − | + | − | + | − | − | + | + | + |
| | PH | − | − | − | − | + | + | + | − | + |
| | PB | − | − | − | − | − | + | − | + | + |
| | n | 10 | 10 | 10 | 10 | 4 | 4 | 10 | 10 | 10 |
| Findings | Grade | ±+2+3+ | ±+2+3+ | ±+2+3+ | ±+2+3+ | ±+2+3+ | ±+2+3+ | ±+2+3+ | ±+2+3+ | ±+2+3+ |

| | | | | |
|---|---|---|---|---|
| Fibrosis | 5 5 | 1 9 | 9 1 | 1 9 10 |
| Vacuolar degeneration | 10 10 | 10 4 | 4 1 9 | 10 10 |
| Ovar cell | 4 6 | 10 | 7 3 | 10 10 |
| Altered hepatocellular foci | 7 3 2 8 | 2 8 4 | 3 1 10 | 1 9 10 |

DEN: diethylnitrosamine, CDAA: choline-deficient L-amino acid-defined diet, PH: partial hepatectomy, PB: phenobarbital, CDAA (−): CRF-1 was fed to animals.

TABLE 4
Number and Area of GST-P Positive Foci

| | | | | | | Number of foci | Focal area |
|---|---|---|---|---|---|---|---|
| Group | DEN | CDAA | PH | PB | n | (/mm²Liver) | (mm²/mm²Liver) |
| 1 | − | − | − | − | 10 | 0.000 ± 0.000 | 0.000 ± 0.000 |
| 2 | − | + | − | − | 10 | 0.035 ± 0.014 CT | 0.013 ± 0.011 CT |
| 3 | + | − | − | − | 10 | 0.350 ± 0.083 CT | 0.006 ± 0.002 CT |
| 4 | + | + | − | − | 10 | 0.332 ± 0.179 ** CT | 0.628 ± 0.112 ** CT |
| 5 | + | − | + | − | 4 | 0.467 ± 0.162 . CT | 0.015 ± 0.006 * CT |
| 6 | + | − | + | + | 4 | 0.640 ± 0.103 ** | 0.017 ± 0.005 * . |
| 7 | + | + | + | − | 10 | 0.231 ± 0.191 * . CT | 0.681 ± 0.148 ** . CT |
| 8 | + | + | − | + | 10 | 0.343 ± 0.202 ** . CT | 0.640 ± 0.121 ** . CT |
| 9 | + | + | + | + | 10 | 0.278 ± 0.141 ** . . . | 0.656 ± 0.094 ** . . . |

DEN: diethylnitrosamine, CDAA choline deficient L-amino acid defined diet, PH: partial hepatectomy, PB. phenobarbital
CDAA(−): CRF-1 was fed to animals.
**,*: Significantly different from control (CT) (**.p < 0.01,*:p < 0.05), -: Not signficantly different

TABLE 5
Number of Foci (/mm²Liver) by Size

| | | | | | | Focal size | Focal size |
|---|---|---|---|---|---|---|---|
| | | | | | | 0-0.03 | 0.03-0.1 |
| Group | DEN | CDAA | PH | PB | n | (mm²) | (mm²) |
| 1 | − | − | − | − | 10 | 0.000 ± 0.000 | 0.000 ± 0.000 |
| 2 | − | + | − | − | 10 | 0.003 ± 0.003 CT | 0.008 ± 0.006 CT |
| 3 | + | − | − | − | 10 | 0.312 ± 0.070 CT | 0.035 ± 0.021 CT |
| 4 | + | + | − | − | 10 | 0.082 ± 0.046 ** CT | 0.079 ± 0.049 ** CT |
| 5 | + | − | + | − | 4 | 0.335 ± 0.124 . CT | 0.105 ± 0.030 ** CT |
| 6 | + | − | + | + | 4 | 0.515 ± 0.088 ** . | 0.098 ± 0.018 ** . |
| 7 | + | + | + | − | 10 | 0.040 ± 0.026 ** . CT | 0.045 ± 0.036 * . CT |
| 8 | + | + | − | + | 10 | 0.110 ± 0.065 ** . CT | 0.072 ± 0.041 ** . CT |
| 9 | + | + | + | + | 10 | 0.075 ± 0.034 ** . * . | 0.047 ± 0.028 ** . . . |

| Focal size | Focal size | Focal size |
|---|---|---|
| 0.1-0.3 | 0.3-1.0 | 1.0-3.0 |
| (mm²) | (mm²) | (mm²) |
| 0.000 ± 0.000 | 0.000 ± 0.000 | 0.000 ± 0.000 |
| 0.011 ± 0.009 CT | 0.012 ± 0.005 CT | 0.002 ± 0.003 CT |
| 0.003 ± 0.005 CT | 0.000 ± 0.000 CT | 0.000 ± 0.000 CT |
| 0.065 ± 0.030 ** CT | 0.054 ± 0.036 ** CT | 0.030 ± 0.022 ** CT |
| 0.023 ± 0.014 . CT | 0.003 ± 0.002 ** CT | 0.000 ± 0.000 . CT |
| 0.022 ± 0.004 ** . | 0.006 ± 0.006 ** . | 0.000 ± 0.000 . . |
| 0.045 ± 0.046 * . CT | 0.046 ± 0.049 . . CT | 0.030 ± 0.033 * . CT |
| 0.064 ± 0.039 ** . CT | 0.047 ± 0.036 * . CT | 0.026 ± 0.017 ** . CT |
| 0.050 ± 0.025 ** . . . | 0.043 ± 0.029 ** . . . | 0.031 ± 0.028 ** . . . |

| | | | | | | Focal size | Focal size |
|---|---|---|---|---|---|---|---|
| | | | | | | 3.0-10.0 | 10.0-30.0 |
| Group | DEN | CDAA | PH | PB | n | (mm²) | (mm²) |
| 1 | − | − | − | − | 10 | 0.000 ± 0.000 | 0.000 ± 0.000 |
| 2 | − | + | − | − | 10 | 0.000 ± 0.001 CT | 0.000 ± 0.000 CT |
| 3 | + | − | − | − | 10 | 0.000 ± 0.000 CT | 0.000 ± 0.000 CT |
| 4 | + | + | − | − | 10 | 0.012 ± 0.007 ** CT | 0.008 ± 0.005 ** CT |
| 5 | + | − | + | − | 4 | 0.000 ± 0.000 . CT | 0.000 ± 0.000 . CT |
| 6 | + | − | + | + | 4 | 0.000 ± 0.000 . . | 0.000 ± 0.000 . . |
| 7 | + | + | + | − | 10 | 0.012 ± 0.013 * . CT | 0.009 ± 0.008 ** . CT |
| 8 | + | + | − | + | 10 | 0.014 ± 0.011 ** . CT | 0.006 ± 0.004 ** . CT |
| 9 | + | + | + | + | 10 | 0.018 ± 0.012 ** . . . | 0.010 ± 0.009 ** . . . |

| | |
|---|---|
| Focal size | Focal size |
| 30.0-100.0 | 100.0- |
| (mm²) | (mm²) |

| | |
|---|---|
| 0.000 ± 0.000 | 0.000 ± 0.000 |
| 0.000 ± 0.000 CT | 0.000 ± 0.000 CT |
| 0.000 ± 0.000 CT | 0.000 ± 0.000 CT |
| 0.002 ± 0.002 ** CT | 0.002 ± 0.002 ** CT |
| 0.000 ± 0.000 . CT | 0.000 ± 0.000 . CT |
| 0.000 ± 0.000 . . | 0.000 ± 0.000 . . |
| 0.002 ± 0.002 ** . CT | 0.001 ± 0.002 ** . CT |
| 0.003 ± 0.002 ** . CT | 0.002 ± 0.002 ** . CT |
| 0.004 ± 0.004 ** . . . | 0.001 ± 0.001 ** . . . |

DEN diethylnitrosamine, CDAA choline-deficient L-amino acid-defined diet, PH partial hepatectomy, PB phenobarbital
CDAA(−) CRF-1 was fed to animals
**.*Significantly different from control (CT) (**p<0.01,*p<0.05). .Not significantly different

What we claimed is:

1. A method for producing primary hepatocellular carcinoma-bearing model animals comprising the following steps;
1) a step for selecting species and strain of model animals in sensitivity on a promotion action for CDAA diet and an sensitivity to the initiation action for DEN, as indexes;
2) a step for administering with DEN under breeding with the CDAA diet to the selected animals and progressing the carcinogenesis and
3) a step for establishing hepatocellular carcinoma-bearing model animals by switching the CDAA diet to a normal diet.

2. The method according to the claim 1, wherein a time period for breeding with the CDAA diet, is at least 64 weeks as a whole.

3. The method according to the claim 1, wherein a time period for breeding with the normal diet, is at least 8 weeks.

4. The method according to the claim 1, wherein the administration of DEN is a one-time administration of carcinogenically effective dose for liver of the selected animals.

5. The method according to the claim 4, wherein the administration of DEN is made after a preliminary breeding time period for a breeding with the CDAA diet.

6. The method according to the claim 5, wherein the preliminary breeding time period is about one week.

7. The method according to the claim 1, wherein the animal species is rat.

8. The method according to the claim 7, wherein a strain of the rat is F344 (Fischer).

9. The method according to the claim 1, wherein the breeding time period with the CDAA diet is at least 64 weeks, the breeding time period with the normal diet is at least 8 weeks and the administration of DEN is a one-time administration of carcinogenically effective dose for a liver of the selected animals.

10. The method according to the claim 9, wherein the selected animals are rats of an F344 (Fischer) strain.

11. The method according to the claim 1, the model animals bearing a different degrees of a hepatocellular carcinoma is produced by controlling the time period for bleeding a choline-deficient amino acid diet after administration of diethylnitrosoamine.

12. The method according to the claim 11, wherein a time period for bleeding the normal diet is at least 8 weeks.

13. A method for diagnosis/therapy of primary hepatocellular carcinoma using the primary hepatocellular carcinoma-bearing model animals produced by the method of any of proceeding the claim 1 to 12.

14. A method of screening diagnostic/preventive/therapeutic agent for primary hepatocellular carcinoma using the primary hepatocellular carcinoma-bearing model animals produced by the method of any of proceeding the claim 1 to 12.

15. Primary hepatocellular carcinoma-bearing model animals produced by the method of the claim 1.

16. The model animals according to the claim 15, wherein said animals bear a different degree of a hepatocellular carcinoma.

17. A novel agent for diagnosis/therapy/prevention for primary hepatocellular carcinoma screened by the method of the claim 14.
